(19) United States
(12) Patent Application Publication
(10) Pub. No.: US 2007/0073339 A1
(43) Pub. Date: Mar. 29, 2007

(54) Anastomosis support device and method

(76) Inventor: Edward Thomas Tirpak, Hamburg, NY (US)

Correspondence Address: Edward T. Tirpak, 163 Brookwood, Hamburg, NY, 14075, US

(21) Appl. No.: 11/235,973

(22) Filed: Sep. 27, 2005

(51) Int. Cl. A61B 17/08; A61D 1/00

(52) U.S. Cl. 606/215; 606/213

(57) ABSTRACT

An implantable device for flexibly supporting an anastomosis and minimizing the risk of blockage of a lumen by external pressures, adhesions, twisting or kinking, and for providing added structural support to minimize the risk and complications of anastomosis failure such as may occur after surgical creation of an anastomosis.

### FIELD OF INVENTION

The present invention relates generally to an implantable device and method for flexibly supporting an anastomosis. The present invention further relates to an implantable support device and method and associated drug or bioactive substance delivery method. The present invention further relates to an implantable support device and method and associated drug or bioactive substance delivery method where the device can be applied intra-lumenally or extra-lumenally and which has a flexible or semi-flexible or elastic construction and design such that the device has (i) sufficient structural strength to inhibit or prevent or minimize the risk of blockage of a lumen by external pressures, adhesions, twisting or kinking, (ii) flexibility to expand or flex as needed to accommodate or partially accommodate lumenal expansion, contraction or constriction, and bending or periostaltic movement, and, where applicable, (iii) sufficient structural rigidity and shape to aid in the alignment of a portion of the lumen in a manner that aids endoscopy such as colonoscopy. The present invention further relates to methods of delivering such implantable device(s) to a lumen and affixing such implantable device(s) to a lumen. The present invention also relates to the targeted administration of one or more drug(s) or biologically active agents or radioactive materials by depositing same on or affixing or otherwise attaching same to such implantable device(s) and introducing such device(s), which may optionally be constructed of biodegradable material, into a living organism in a manner that provides for localized placement and administration of such one or more drug(s) or biologically active agents or radioactive materials.

### BACKGROUND

The prior art includes a wide variety of stents and grafts. For example, Palmaz, U.S. Pat. No. 4,733,655, discloses a balloon expandable intra-lumenal graft, including an embodiment comprising a wire mesh tube. Intersecting wire members, secured to one another at there intersections by welding, soldering or gluing, form the wire mesh and form a diamond-like pattern. This structure provides a relatively high resistance to radial collapse, but it suffers from a number of disadvantages. First, it is a rigid structure which cannot easily assume the configuration of a curved vessel or lumen which receives it. Second, one must use a balloon catheter to expand and implant it. This requirement and its rigidity limit aspects of the Palmaz graft such as its potential length and the type of lumen it may safely be applied to.

Other prior stents have a more flexible construction, but they suffer from other disadvantages. Wiktor, U.S. Pat. No. 4,886,062, for example, discloses a stent which has a relatively flexible construction. This construction includes a deformable wire bent in a zig-zag design and coiled in a spiral fashion. The resulting wire tube has an open configuration with a reduced hoop strength. Each hoop lies essentially isolated from adjacent hoops and does not obtain substantial support from them. Moreover the open configuration increases the risk that plaque elements may herniated through the coil. Finally, one must use a balloon catheter to expand and implant it. Thus there are resulting limitations such as the Wiktor device length having to be no longer than the balloon catheter.

The embodiments of the present invention are more broadly applicable than the prior art stents and grafts and avoid their associated disadvantages. Among the embodiments of the present invention are one or more devices that have a flexible or elastic construction that allows them to mold to, conform with and follow the curvature of the vessel or lumen they are affixed to. Among the embodiments of the present invention are one or more devices that can be affixed to the exterior of a lumen thus avoiding potential risks associated with affixing devices to the interior of a lumen within living organisms.

Examples of technological obstacles addressed by the present invention and the several embodiments of the present invention include, without limitation, (i) partial or complete blockage of the small bowel or large bowel of living organisms resulting from kinking, twisting at or near an anastomisis site or from the formation of adhesions post-surgery, (ii) limitations on the physical range and usefulness of post-surgical endoscopy, such as, without limitation, endoscopy at, within or past an anastomosis site or within the small bowel of living organism or for example, at, within or past the juncture of the small bowel and large bowel if that is the location of the anastomosis site, and (iii) the relative unavailability of optimal modalities for localized administration or time released administration of drugs or other bioactive materials or radioactive materials.

### SUMMARY OF THE INVENTION

In accordance with some preferred embodiments of the present invention and intra-lumenal or extra-lumenal device comprises a device formed of or created from biocompatible materials and constructed, molded or otherwise created to have (i) a conical or semi-conical shape at one end, (ii) one or more portion(s) at the other end shaped to fit a lumen, said portion(s) most preferably longitudinal in shape, where, if more than one such end portions are present, such more than one such end portions being separate from each other most preferably longitudinally, and (iii) an overall cylindrical, semi-cylindrical or other shape configured to fit in or on a lumen where such device has an actual or potential gap extending longitudinally along the device such that at no point on the device is there a lateral fixed continuous circumference or where, even if one or more portion(s) of the device contact(s) another or other portion(s) of the device so that a complete lateral circumference is present, such contacting portions can be separated from or moved relative to each other through bending, expansion, contraction or other flexing of the device. For simplicity of description the meaning of “lumen” as used herein is intended to include, without limitation, one or more tubular organ(s), such as for example an intestine or intestines, in addition to meaning the space within a tubular organ.

In accordance with other preferred embodiments of the present invention an extra-lumenal or intra-lumenal device comprises a device formed of or created from biocompatible materials and constructed, molded or otherwise created to have (i) one or more portion(s) at either or both end(s) with said portion(s) shaped to fit a lumen, most preferably longitudinal in shape, where, if more than one such end portions are present, such more than one such end portion(s) at one or both ends being separate from each other most preferably longitudinally and (ii) an overall cylindrical, semi-cylindrical or other shape configured to fit in or on a lumen where such device has an actual or potential gap extending longitudinally along the device such that at no point on the device is there a lateral fixed continuous circumference or where, even if one or more portion(s) of the device contact(s) another or other portion(s) of the device so that a complete lateral circumference is present, such contacting portions can be separated from or moved relative to each other through bending, expansion, contraction or other flexing of the device.

In accordance with other preferred embodiments of the present invention an extra-lumenal or intra-lumenal device comprises a device formed of or created from biocompatible materials and constructed, molded or otherwise created to have an overall generally cylindrical, semi-cylindrical or other shape configured to fit in or on a lumen where such device has an actual or potential gap extending longitudinally along the device such that at no point on the device is there a lateral fixed continuous circumference or where, even if one or more portion(s) of the device contact(s) another or other portion(s) of the device so that a complete lateral circumference is present, such contacting portions can be separated from or moved relative to each other through bending, expansion, contraction or other flexing of the device.

In accordance with other preferred embodiments of the present invention any one or more of the device(s) or embodiment(s) of the invention described herein further comprising such device(s) or embodiment(s) being separately affixed in or on a single lumen, most preferably laterally to each other, such that said device(s) or embodiment(s) are not in physical contact with each other or alternatively such that said device(s) or embodiment(s) are in contact with each other.

In accordance with other preferred embodiments of the present invention any one or more of the device(s) or embodiment(s) of the invention described herein further comprising such device(s) or embodiment(s) being affixed, attached or otherwise connected to a lumen or to one or more other of the device(s) or embodiment(s) of the invention described herein by or via other device(s), clip(s), clamp(s), adhesive(s), mesh or wire or other materials, bands or other method(s), device(s) or mechanisms of any nature or manner.

In accordance with other preferred embodiments of the present invention any one or more of the device(s) or embodiment(s) of the invention described herein further comprising such device(s) or embodiment(s) having one or more drugs and/or other bio-active agents or materials and/or radioactive materials and/or biodegradable materials adhered thereupon, affixed thereupon and/or embedded on or in such device(s) or embodiment(s).

In accordance with other preferred embodiments of the present invention any one or more of the device(s) or embodiment(s) of the invention described herein further comprising such device(s) or embodiment(s) having been constructed of a significant amount of, or entirely of, biodegradable materials, such as for example where the device(s) or embodiment(s) are made up of in excess of about one percent (1%) by weight of biodegradable materials.

An aspect of the invention and the embodiments described herein is the methods of making and using any one or more of the device(s) or embodiments of the invention.

In accordance with other preferred embodiments of the present invention any one or more of the device(s) or embodiment(s) of the invention described herein further comprising such device(s) or embodiment(s) having been constructed partially or entirely of any flexible or semi-flexible material or design configuration such as for example wire mesh of metal or other material(s) or molded plastic or composite material(s) and/or where varying materials and/or thicknesses of materials or shapes or configurations of materials are within or part of such device(s) or embodiment(s).

An aspect of the invention and the embodiments described herein is that such device(s) or embodiments of the invention described herein would (i) mitigate the risk of for example (i) partial or complete blockage of the small bowel or large bowel of living organisms resulting from kinking, twisting at or near an anastomisis site or from the formation of post-surgical adhesions, (ii) provide structural support at or near an anastomosis to minimize the risk of anastomosis failure, (iii) aid in overcoming limitations on the physical range and usefulness of post-surgical endoscopy, such as, without limitation, endoscopy at, within or past an anastomosis site or within the small bowel of living organism or for example, at, within or past the juncture of the small bowel and large bowel if that is the location of the anastomosis site, or (iv) enhance the availability of optimal modalities for localized administration or time released administration of drugs or other bioactive materials or radioactive materials.

It should be understood that the aforesaid Summary of the Invention and any and all descriptions, examples and disclosures herein are intended to be descriptive and explanatory and are not intended to in any way limit or be restrictive of the invention as claimed. Unless specified to the contrary standard techniques and methods and parameters are to be used for the present invention. Unless specified otherwise any procedures, techniques, parameters, specifications and conditions are intended to be approximate. All ranges specified herein are intended to encompass the entire range and any portion or sub-range within the specified range. Procedures, techniques, parameters, specifications and conditions provided within the Examples are intended to be specific, not approximate. However, other equivalent procedures, techniques, parameters, specifications and conditions can, of course, also be used.

### BRIEF DESCRIPTION OF THE DRAWINGS

FIGS. 1A-1B show views of embodiments of the present invention having a generally conical or semi-conical shape at one end, one or more portion(s) at the other end elongated in shape, where such elongated end portions are separated from each other longitudinally, and an overall cylindrical or semi-cylindrical shape configured to fit in or on a lumen where such device has an actual or potential gap extending longitudinally along the device. Each figure shows a three dimensional view and a flattened more two dimensional view.

FIGS. 2A-2B show views of embodiments of the present invention having one or more portion(s) at both ends elongated in shape, where such elongated end portions are separated from each other longitudinally, and having an overall cylindrical or semi-cylindrical shape configured to fit in or on a lumen where such device(s) have an actual or potential gap extending longitudinally along the device(s). Each figure shows a three dimensional view and a flattened more two dimensional view.

FIGS. 3A-3D show views of embodiments of the present invention having a generally elongated structure with varied shapes. Each figure shows a three dimensional view and a flattened more two dimensional view.

FIG. 4 shows embodiments of the present invention having a generally elongated structure independently connected to each a lumen across an anastomosis.

FIG. 5 shows embodiments of the present invention having a generally elongated structure connected to each other. The figure shows a three dimensional view and a flattened more two dimensional view.

### DETAILED DESCRIPTION OF THE INVENTION

Numerous embodiments of the present invention relate to an implantable device for flexibly supporting an anastamosis in a lumen. By having portions of such device(s) embodying one or more embodiment(s) of the invention, and most preferably flexible portion(s) of said device(s), positioned on and affixed to a lumen, such as for example a small intestine within a mammal, such that an anastamosis in such lumen is partially or entirely spanned by such device(s) longitudinally along said lumen and by having said device(s) constructed, shaped or otherwise made to allow for flexible or semi-flexible bending and constriction at or near said anastomosis site said device(s) would allow for normal or semi-normal functioning of said lumen while simultaneously, by adding some structural support, and most preferably flexible structural support, minimizing the risk of excessing bending, kinking or twisting of said lumen that could block, obstruct or otherwise interfere with the normal functioning of said lumen, and would also aid structural support, most preferably flexible structural support to lessen the risk of anastomosis failure and the associated risks and harm associated with a lumen opening at an anastamosis site.

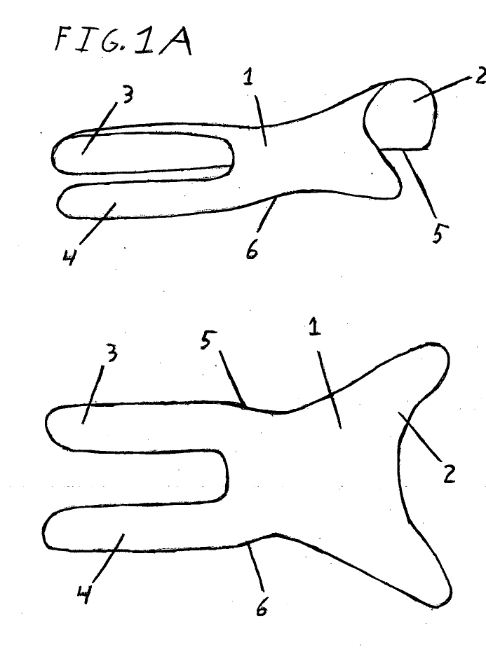

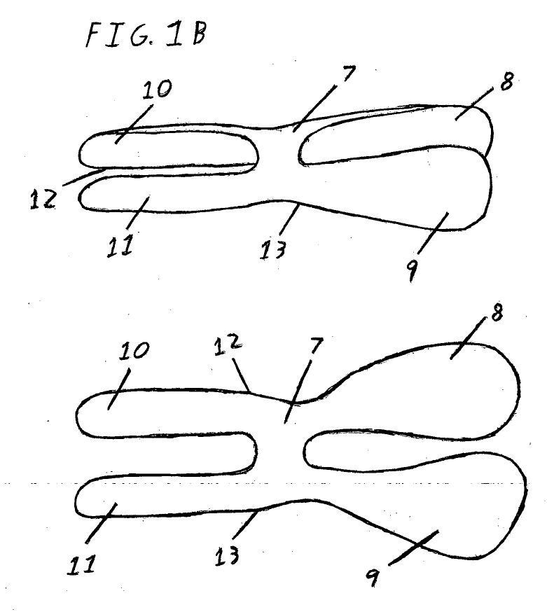

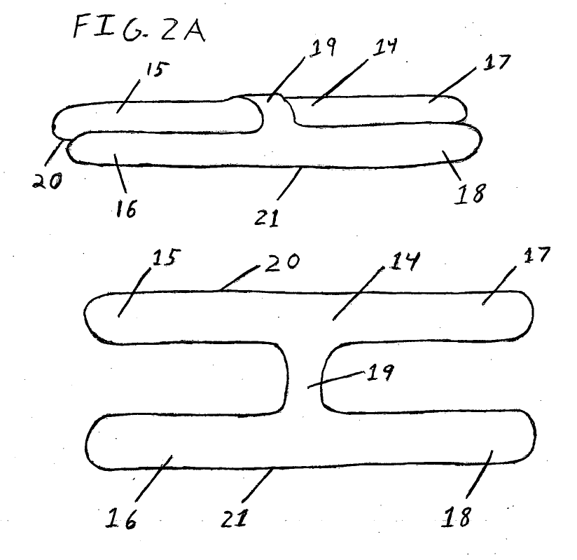

One preferred embodiment of the invention relates to an implantable device for flexibly supporting an anastomosis such as the embodiment depicted in FIG. 1A where the device 1 has at one end a generally conical or semi-conical shape 2 and at the other end one or more and most preferably two elongated end portions separated from each other longitudinally 3 and 4 and where longitudinal edges 5 and 6 are configured to provide a gap between such longitudinal edges 5 and 6 when the device is affixed to a lumen and where the device 1 has a relatively larger diameter at the conical portion 2 that fits with, in or upon the portion of such lumen having a relatively larger diameter or circumference and other portions 3 and 4 that fit with in or on a lumen or portion of the lumen having a comparatively smaller diameter or circumference. For this and other embodiments of the present invention biocompatible material(s) is or are shaped, constructed, molded or otherwise made in such a way as to provide a size, shape and structure that provides flexible support to a luminal structure having an anastomosis and to which such device or embodiment will be attached or otherwise affixed. This embodiment of the invention would provide structural support at or near an anastamosis site most particularly where an anastamosis is at the juncture of two lumens having different sizes or diameters relative to each other such as for example where an anastamosis is at the juncture of the small and large intestines of a mammal. Said structural support provided by this embodiment at said juncture of a mammal would also provide structural support for the alignment of endoscopy equipment or device(s) at, within or through the anastomosis. Another variation of this preferred embodiment involves the generally conical or semi-conical shape 2 at one end of the device being elongated in at least one portion such that for example two elongated portions are present resembling generally larger versions of the smaller elongated members 3 and 4 at the other end of the device.

Other preferred embodiments of the invention also relate to an implantable device for flexibly supporting an anastomosis such as the embodiment depicted in FIG. 1B where the device 7 has at one end one or more generally elongated portions 8 and 9, where if more than one such elongated portion is present they are separated from each other longitudinally, and at the other end one or more generally elongated portions 10 and 11, where if more than one such elongated portion is present they are separated from each other longitudinally, where longitudinal edges 12 and 13 are configured to provide a gap between such longitudinal edges 12 and 13 and where the device 7 has a relatively larger diameter at the elongated portions 8 and 9 that fit with, in or upon or accommodates a portion of a lumen having a relatively larger diameter or circumference and a comparatively smaller diameter at the elongated portions 10 and 11 that fit with, in or on a lumen or portion of a lumen having a comparatively smaller diameter or circumference. This embodiment of the invention would provide structural support at or near an anastamosis site most particularly where an anastamosis is at the juncture of two lumens having different sizes or diameters relative to each other such as for example where an anastamosis is at the juncture of the small and large intestines of a mammal.

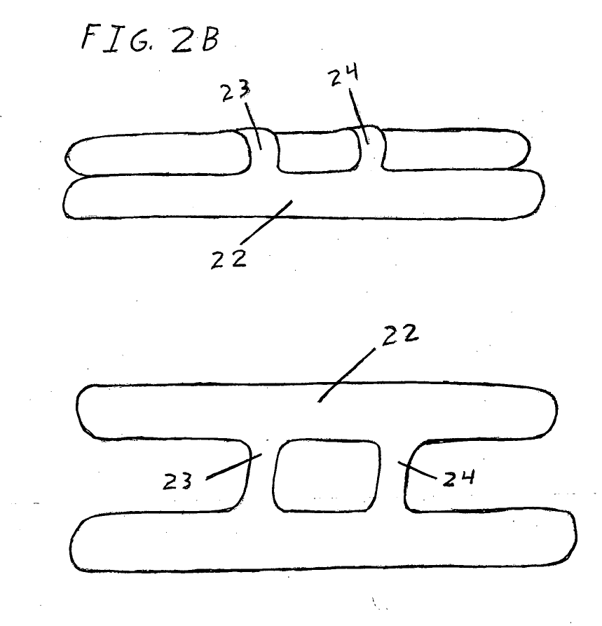

Other preferred embodiment(s) of the invention also relate to an implantable device for flexibly supporting an anastomosis such as the embodiment depicted in FIG. 2A where the device 14 is formed of or created from biocompatible materials and constructed, molded or otherwise created to have one or more elongated end portion(s) at either or both end(s) 15, 16, 17 and 18 with the device 14 most preferably shaped to fit or accommodate a lumen and said end portion(s) 15, 16, 17 and 18 also most preferably shaped to fit or accommodate a lumen, and said end portion(s) 15, 16, 17 and 18 most preferably being elongated, and where, if more than one such end portion(s) 15, 16, 17 and 18 are present on an end of the device 14, such more than one such end portion(s) 15, 16, 17 and 18 at such end of the device 14 are separate from any other such end portion(s) 15, 16, 17 and 18 at that end of the device 14 most preferably longitudinally and the device 14 has an overall generally cylindrical, semi-cylindrical or other shape with a connecting portion 19 configured to fit in or on or otherwise accommodate a lumen where such device 14 has an actual or potential gap extending longitudinally along the edges of the device 20 and 21 such that at no point on the device 14 is there a lateral fixed continuous circumference or where, even if one or more portion(s) of the device for example at or near the edges 20 and 21 contact(s) another or other portion(s) of the device so that a complete lateral circumference is present, such contacting portions can be separated from or moved relative to each other through bending, expansion, contraction or other flexing of the device 14. The elongated portions at one end of the device may be shaped or molded to fit a lumen having a one diameter while the elongated portions at the other end of the device may be shaped or molded to fit a lumen or portion of a lumen having a different diameter or the same diameter as may be appropriate to fitting the device to the portions of a lumen on either side of an anastomosis. FIG. 2B shows a device 22 generally similar to the device 14 shown in FIG. 2A but having two connecting portions 23 and 24. Embodiments of the invention may have any number of connecting portions such as 23 and 24.

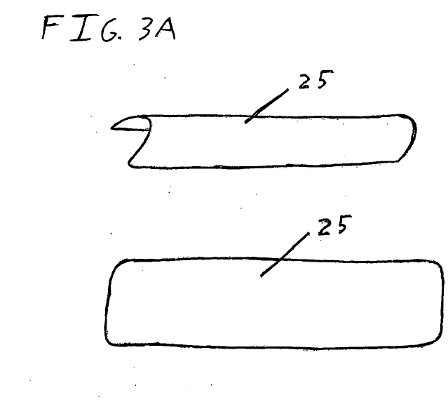

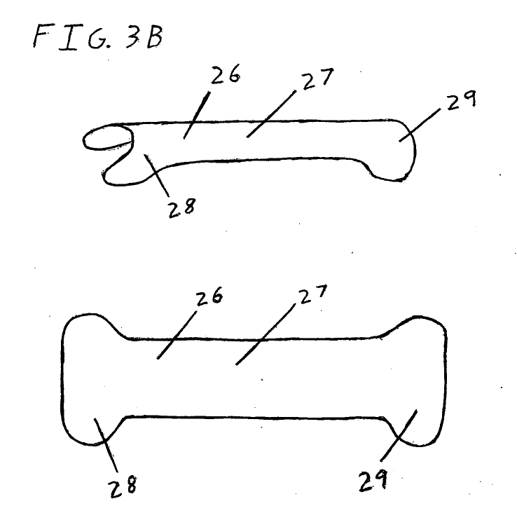

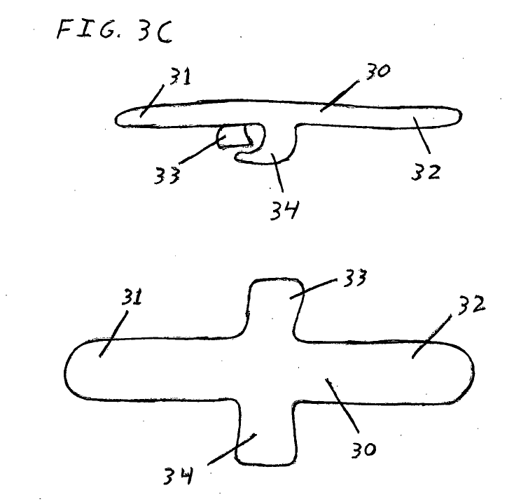

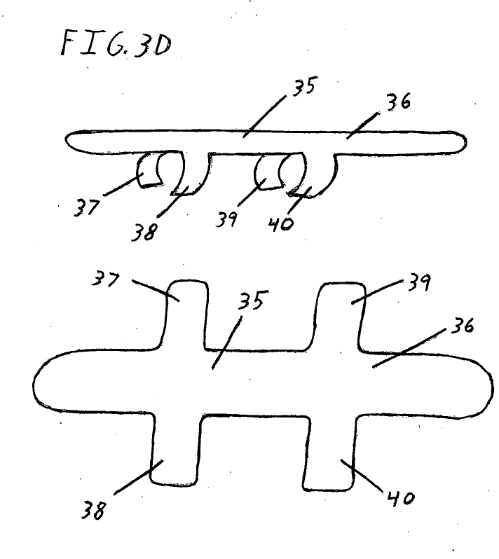

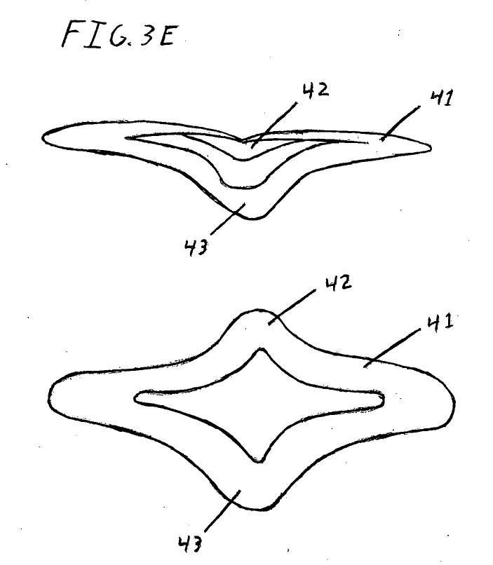

Other preferred embodiment(s) of the invention also relate to an implantable device for flexibly supporting an anastomosis such as the embodiment depicted in FIG. 3A where the device 25 is formed of or created from biocompatible materials and constructed, molded or otherwise created to have an overall generally cylindrical, semi-cylindrical or other shape configured to fit in or on a lumen where such device has an actual or potential gap extending longitudinally along the device such that at no point on the device is there a lateral fixed continuous circumference or where, even if one or more portion(s) of the device contact(s) another or other portion(s) of the device so that a complete lateral circumference is present, such contacting portions can be separated from or moved relative to each other through bending, expansion, contraction or other flexing of the device. This preferred embodiment as well as others may also be formed or created as a strip or other shape or structure that is affixed to a lumen in such a way that such strip or other shape extends partly or fully across an anastomosis. FIG. 3B shows a device 26 having a thin middle section 27 and broader end portions 28 and 29. FIG. 3C shows a device 30 having elongated ends 31 and 32 and two rounded curved brace portions 33 and 34 near the middle of the device 30. FIG. 3D shows a device 35 having an elongated body 36 and two rounded curved brace portions 37 and 38 located across the body 36 from each other at one portion of the device 35 and another two rounded curved brace portions 39 and 40 located across the body 36 from each other at a different location on the device 35. FIG. 3E shows a device 41 having an elongated diamond shape, although round, oval or other shapes are contemplated as well, with rounded corners and an aperture in the center which device 41 is curved such that two apposing corners 42 and 43 are curved toward each other so the overall shape of the device is cylindrical or semi-cylindrical and/or would fit on, around or otherwise accommodate a lumen.

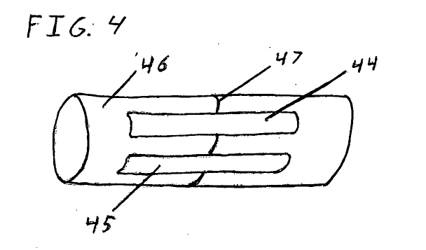

Other preferred embodiment(s) of the invention also relate to an implantable device for flexibly supporting an anastomosis such as the embodiment depicted in FIG. 4 where one or more device(s) 44 and 45 are separately affixed in or on a single lumen 46, most preferably laterally to each other and across an anastomois 47, such that said device(s) 44 and 45 are not in physical contact with each other, or, alternatively, such that said device(s) 44 and 45 are in contact with each other but can be moved relative to each other through bending, expansion, contraction or other flexing of the device(s) 44 and 45 or the lumen 46.

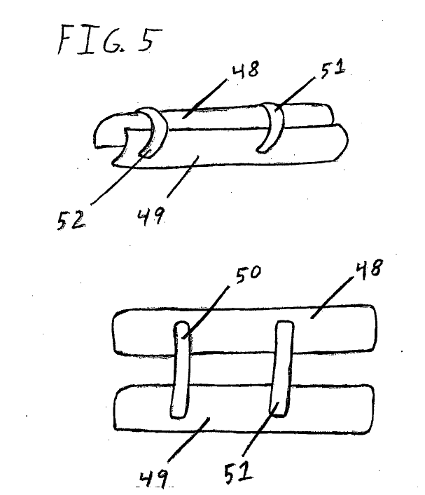

Other preferred embodiment(s) of the invention also relate to an implantable device for flexibly supporting an anastomosis such as the embodiment depicted in FIG. 5 where one or more of the device(s) 48 and 49 are affixed, attached or otherwise connected to a lumen or to one or more other of the device(s) 48 and 49 by or via other device(s), clip(s). 50 and 51, clamp(s), adhesive(s), mesh of wire or other materials, bands, or other method(s), device(s) or mechanisms of any nature or manner.

Other preferred embodiment(s) of the invention also relate to an implantable device for flexibly supporting an anastomosis such as the embodiment depicted in any of the figures where one or more of the device(s) has one or more structure(s), element(s) or component(s) of any type such as for example flexible mesh or artificial membrane that bridge(s), span(s) or otherwise connect(s) the aspect(s) or component(s) or portion(s) or part(s) of any one or more of the device(s) or embodiment(s) across any actual or potential gap extending longitudinally along such device or embodiment such that there is a lateral continuous circumference. For such embodiment(s) said one or more structure(s), element(s) or component(s) of any type would most preferably be flexible and elastic in nature to accommodate bending, expansion, contraction or other flexing of the device(s).

Other preferred embodiment(s) of the invention also relate to an implantable device for flexibly supporting an anastomosis such as the embodiment depicted in FIG. 2B where the device 14 has one or more drugs and/or other biologically active agents or materials and/or radioactive materials and/or biodegradable materials adhered thereupon, affixed thereto and/or embedded on or in such device(s).

Other preferred embodiment(s) of the invention also relate to an implantable device for flexibly supporting an anastomosis such as the embodiment depicted in FIG. 2B where one or more embodiment(s) of the invention are constructed of a significant amount of biodegradable materials, such as for example where the device(s) or embodiment(s) are made up of in excess of about one percent (1%) by weight of biodegradable materials. Any and all possible weight percent concentrations and combinations of biodegradable material(s) suitable for implantation within a human or other mammal may be used.

Other preferred embodiment(s) and aspect(s) of the invention also relate to methods of making and using any one or more of the device(s) or embodiments of the invention described herein.

Other preferred embodiment(s) of the invention also relates to any one or more of the device(s) or embodiment(s) of the invention described herein further comprising such device(s) or embodiment(s) being constructed partially or entirely of any flexible or semi-flexible material or design configuration such as for example wire mesh of metal or other material(s) or molded plastic or composite material(s) and/or where varying or multiple materials and/or thicknesses of materials or shapes or configurations of materials are within or part of such device(s) or embodiment(s). Emdodiments of the invention may optionally be constructed to be flat or semi-flat or other shape prior to application to a lumen or pre-molded to another or more cylindrical shape more consistent with a lumen shape prior to application of the embodiment device to a lumen.

Materials and techniques and methods for making implantable medical devices are well established and known to persons having ordinary skill in the art, and such materials, techniques and methods can be used in constructing, molding or creating or making and using embodiments of the invention. Materials suitable for making and using one or more embodiment(s) of the present invention include, without limitation, biocompatible metal(s) or alloy(s), biocompatible composite material(s) or plastic(s) or polymer(s) and non-biocompatible materials covered with or encased within biocompatible materials. Methods suitable for creation of or making or using one or more embodiment(s) of the invention include, without limitation, the standard techniques used in the creation and use of implantable stents and grafts of metal or other materials, with or without drug(s), biologically active material(s) or radioactive material(s) affixed or attached to such stents or grafts or embedded within such stents or grafts, standard methods and techniques used in the creation and use of implantable prostheses, pace-makers or other devices suitable for implantation in humans or other mammals, and standard methods and techniques used in creating and using molded plastic or composite or polymer based or similar items suitable for implantation in humans or other mammals, and any standard methods and techniques and materials used in affixing drug(s), biologically active material(s) or radioactive material(s) to such plastic or composite or polymer based or similar items or materials, and standard methods and techniques and materials used in coating or encasing materials, devices or other items with or in, respectively, biocompatible material(s). The embodiments of the invention may have perforations, holes or apertures suitable for aiding in affixing such embodiments to a lumen. The following United States Patents disclose materials and methods and techniques useful in making and using elements or components of one or more embodiment(s) of the present invention and are hereby incorporated by reference, U.S. Pat. No. 6,945,991, U.S. Pat. No. 6,863,684, U.S. Pat. No. 6,652,575, U.S. Pat. No. 6,852,122, U.S. Pat. No. 6,884,212, U.S. Pat. No. 6,833,408, U.S. Pat. No. 4,733,655, U.S. Pat. No. 4,866,062 and U.S. Pat. No. 6,939,372.

Later developed materials and techniques and methods suitable for making one or more embodiment(s) of the present invention are intended to be incorporated into the practice and use of the current invention and are not intended to be in any way limiting on the scope or intended uses or practice of the present invention.

The embodiments of the invention may be used in numerous varying ways while still maintaining meaningful utility. One, non-inclusive, method of using the embodiments of the invention involves during surgery on a human or other mammal where a component of such surgery is the creation of an anastamosis, such as for example a small bowel resection or hemi-colectomy, having qualified medical personnel apply said embodiment on or around the lumen having the anastamosis such that the device embodying the invention (“Sample Device”) extends longitudinally along said lumen onto or across said anastamosis and position and/or affix said Sample Device to said lumen using suitable bioadhesive substance(s) or sutures or other method(s), which bioadhesive substances could be applied to the Sample Device at any time prior to the affixing or positioning of said Sample Device or applied to the lumen itself, or any other method suitable for affixing or attaching or positioning an implantable device within a human or other mammal.

If the Sample Device is an embodiment of the invention having one or more drug(s), biologically active material(s) or radioactive material(s) affixed to or as a component of such Sample Device, such drug(s), biologically active material(s) or radioactive material(s) would most preferably be loaded onto or within or affixed to such Sample Device in a manner and at a time best suited to such drug(s)', biologically active material(s)' or radioactive material(s)' intended use(s) and timing and mode of delivery or administration as would be known to those having ordinary skill in the art. Standard methods and techniques for loading, affixing or including drug(s), biologically active material(s) or radioactive material(s) to, on or in implantable devices such as implantable stents would most preferably be used for loading, affixing or including drug(s), biologically active material(s) or radioactive material(s) to, on or in embodiments of the present invention. U.S. Pat. No 6,946,146 discloses materials and methods and techniques useful in making and using one or more embodiment(s) of the present invention and is hereby incorporated by reference. Embodiments of the present invention may also be assembled into a surgical kit for use in a procedure for flexibly supporting an anastomosis in a lumen with the surgical kit comprising an implantable device in the form of one or more embodiments of the present invention and having a shape configured to the exterior of a lumen, at least one securement means for securing the implantable device to tissue such as for example bioadhesive materials or sutures, and wherein the implantable device is sterile packaged.

The methods and techniques described herein are intended to be instructive and not to in any way limit the scope of the invention or any associated methods of use of the invention. Any methods suitable to the implantation or positioning or affixing or attaching of implantable medical devices may be used with the present invention.

### EXAMPLES

### Example 1

One example of the present invention is the embodiment depicted in FIG. 1A where the device 1 is approximately eight centimeters in total length and approximately 0.5 millimeters to approximately 4 millimeters thick, has at one end a generally conical or semi-conical shaped portion 2 that has an unflexed diameter of one inch at its widest point and at the other end of the device 1 two elongated end portions 3 and 4 each approximately 3.25 centimeters in length separated from each other longitudinally and such that the unflexed lateral distance between such elongated end portions 3 and 4 is approximately one centimeter and where longitudinal edges 5 and 6 are configured to provide a gap between such longitudinal edges 5 and 6 along the entire device 1. For this example embodiment the device is constructed of a flexible non-metalic material molded into a preformed shape that because of the shape and material used provides for bending and partial twisting of the device 1 and the lateral expansion and contraction of the device 1 along its entire length or at one or more points along its length.

### Example 2

Another example of the present invention is the embodiment depicted in FIG. 1A where the device 1 is approximately eight centimeters in total length and approximately 0.5 millimeters to approximately 4 millimeters thick, has at one end a generally conical or semi-conical shaped portion 2 that has an unflexed diameter of two centimeters at its widest point but where said portion 2 is elongated in two places such that a curved elongated lobe portion is present on either side of the said portion 2 and at the other end of the device 1 two elongated end portions 3 and 4 each approximately 3.25 centimeters in length separated from each other longitudinally and such that the unflexed lateral distance between such elongated end portions 3 and 4 is one centimeter and where longitudinal edges 5 and 6 are configured to provide a gap between such longitudinal edges 5 and 6 along the entire device 1. For this example embodiment the device is constructed of a flexible non-metalic material molded into a preformed shape that because of the shape and material used provides for bending and partial twisting of the device 1 and the lateral expansion and contraction of the device 1 along its entire length or at one or more points along its length.

### Example 3

Another example of the present invention is the embodiment depicted in FIG. 2A where the device 14 is approximately eight centimeters in total length and approximately 0.5 millimeters to approximately 4 millimeters thick, has a flexible bracing portion 19, has at both ends two elongated end portions, 15 and 16 on one end and 17 and 18 on the other end, each such end portion being approximately 3.25 centimeters in length separated from the other on the same end longitudinally and such that the unflexed lateral distance between such elongated end portions 15 and 16 is approximately one centimeter and the unflexed lateral distance between such elongated end portions 17 and 18 is also approximately one centimeter and where longitudinal edges 20 and 21 are configured to provide a gap between such longitudinal edges 20 and 21 along the entire device 14. For this example embodiment the device is constructed of a flexible non-metalic material molded into a preformed shape that because of the shape and material used provides for bending and partial twisting of the device 14 and the lateral expansion and contraction of the device 14 along its entire length or at one or more points along its length. Another example would be similar but constructed of a different material such as flexible wire mesh.

### Example 4

Another example of the present invention is the embodiment depicted in FIG. 3A where the device 25 is approximately eight centimeters in total length and approximately 0.5 millimeters to approximately 4 millimeters thick, has a generally cylindrical shape with rounded corners and an unflexed lateral width of approximately one centimeter. For this example embodiment the device is constructed of a flexible non-metalic material molded into a preformed shape that because of the shape and material used provides for bending and partial twisting of the device 25 and the lateral expansion and contraction of the device 25 along its entire length or at one or more points along its length. Another example would be similar but constructed of a different material such as flexible wire mesh.

### Example 5

Another example of the present invention is the invention described in Example 4 but where the unflexed lateral width of the specified device 25 is approximately 0.5 centimeters and two such devices are affixed to a lumen in a manner such that each of the two devices spans a single anastomosis in a lumen longitudinally and each of the two devices is positioned on the opposite side of the lumen relative to the other such device. For this example embodiment the device is constructed of a flexible non-metalic material molded into a preformed shape that because of the shape and material used provides for bending and partial twisting of the device 25 and the lateral expansion and contraction of the device 25 along its entire length or at one or more points along its length. Another example would be similar but constructed of a different material such as flexible wire mesh.

### Example 6

Another example of the present invention is the invention described in Example 5 but additionally having a connecting device such as a metal clip, non-metalic or other connecting device linking or attaching each said device to the other. For this example embodiment the device is constructed of a flexible non-metalic material molded into a preformed shape that because of the shape and material used provides for bending and partial twisting of the device 25 and the lateral expansion and contraction of the device 25 along its entire length or at one or more points along its length. Another example would be similar but constructed of a different material such as flexible wire mesh.

### Example 7

Another example of the present invention is the invention described in Example 3 but where the interior or exterior of the device 14 has affixed thereto, adhered thereupon or embedded therein one or more drug(s), biodegradable materials, biologically active agents or materials and/or radioactive materials. For this example embodiment the device is constructed of a flexible non-metalic material molded into a preformed shape that because of the shape and material used provides for bending and partial twisting of the device 14 and the lateral expansion and contraction of the device 14 along its entire length or at one or more points along its length. Another example would be similar but constructed of a different material such as flexible wire mesh.

What is claimed is:

1. An implantable device for flexibly supporting an anastomosis comprising a flexible device shaped to fit on the exterior of a lumen across an anastomosis.

2. The device of claim 1 further comprising said device having at least one biologically active material or drug or biodegradable material affixed to said device.

3. The device of claim 1 further comprising said device having at one end a generally conical shape and said device having at the other end at least one elongated end portion.

4. The device of claim 3 further comprising said device having at least one biologically active material or drug or biodegradable material affixed to said device.

5. The device of claim 1 further comprising said device having at one end a generally conical shape and said device having at the other end at least two elongated end portions.

6. The device of claim 5 further comprising said device having at least one biologically active material or drug or biodegradable material affixed to said device.

7. The device of claim 1 further comprising said device having at one end at least two elongated end portions and at the other end at least two elongated end portions with the elongated end portions at one end of said device being a different size than the elongated end portions at the other end of said device.

8. The device of claim 7 further comprising said device having at least one biologically active material or drug or biodegradable material affixed to said device.

9. The device of claim 1 further comprising said device having at one end at least one elongated end portion and at the other end at least one elongated end portion.

10. The device of claim 9 further comprising said device having at least one biologically active material or drug or biodegradable material affixed to said device.

11. The device of claim 1 further comprising said device having at one end at least two elongated end portions and at the other end at least two elongated end portions.

12. The device of claim 11 further comprising said device having at least one biologically active material or drug or biodegradable material affixed to said device.

13. The device of claim 1 further comprising said device having at one end at least two elongated end portions and at the other end at least two elongated end portions and said device having at least one connecting portion.

14. The device of claim 13 further comprising said device having at least one biologically active material or drug or biodegradable material affixed to said device.

15. The device of claim 1 further comprising said device having at least one curved brace portion.

16. The device of claim 15 further comprising said device having at least one biologically active material or drug or biodegradable material affixed to said device.

17. The device of claim 1 further comprising said device having an elongated diamond shape with rounded corners and an aperture in the center.

18. The device of claim 17 further comprising said device having at least one biologically active material or drug or biodegradable material affixed to said device.

19. The device of claim 1 further comprising said device having at least two portions independently affixed to a lumen across an anastomosis.

20. The device of claim 19 further comprising said device having at least one biologically active material or drug or biodegradable material affixed to said device.

21. The device of claim 1 further comprising said device having at least two portions attached to each other by at least one clip, clamp, adhesive, mesh, wire, material, band or other mechanisms.

22. The device of claim 21 further comprising said device having at least one biologically active material or drug or biodegradable material affixed to said device.

23. A method of flexibly supporting an anastomosis comprising affixing to the exterior of a lumen a flexible implantable device across an anastomosis.

24. The method of claim 23 further comprising said device having at one end a generally conical shape and said device having at the other end at least one elongated end portion.

25. The method of claim 23 further comprising said device having at one end a generally conical shape and said device having at the other end at least two elongated end portions.

26. The method of claim 23 further comprising said device having at one end at least two elongated end portions and at the other end at least two elongated end portions with the elongated end portions at one end of said device being a different size than the elongated end portions at the other end of said device.

27. The method of claim 23 further comprising said device having at one end at least one elongated end portion and at the other end at least one elongated end portion.

28. The method of claim 23 further comprising said device having at one end at least two elongated end portions and at the other end at least two elongated end portions.

29. The method of claim 23 further comprising said device having at one end at least two elongated end portions and at the other end at least two elongated end portions and said device having at least one connecting portion.

30. The method of claim 23 further comprising said device having at least one curved brace portion.

31. The method of claim 23 further comprising said device having an elongated diamond shape with rounded corners and an aperture in the center.

32. The method of claim 23 further comprising said device having at least two portions independently affixed to a lumen across an anastomosis.

33. The method of claim 23 further comprising said device having at least two portions attached to each other by at least one clip, clamp, adhesive, mesh, wire, material, band or other mechanisms.

34. A surgical kit for use in a procedure for flexibly supporting an anastomosis, the surgical kit comprising: an implantable device having a shape configured to the exterior of a lumen, securement means for securing the implantable device to tissue, and wherein the preassembled implantable article is sterile packaged.
